(19) United States
(12) Patent Application Publication
(10) Pub. No.: US 2025/0225289 A1
(43) Pub. Date: Jul. 10, 2025

(54) DESIGN DEVICE, DESIGN METHOD, PROGRAM, POROUS STRUCTURE, AND MANUFACTURING METHOD THEREFOR

(71) Applicant: National University Corporation Hokkaido University, Hokkaido (JP)

(72) Inventors: Satoshi Yamada, Hokkaido (JP); Hayato Suzuki, Hokkaido (JP); Masahiro Todoh, Hokkaido (JP); Kazuki Sawada, Hokkaido (JP); Akihiko Nishimura, Hokkaido (JP)

(21) Appl. No.: 18/853,056

(22) PCT Filed: Mar. 29, 2023
(86) PCT No.: PCT/JP2023/012987

§ 371 Date: Sep. 30, 2024

(30) Foreign Application Priority Data
Mar. 31, 2022 (JP) 2022-058004

(51) Int. Cl. G06F 30/18

(52) U.S. Cl. CPC G06F 30/18

(57) ABSTRACT

A design device (100) includes: a beam generator (152) that repeatedly generates a plurality of new beams branching from a node closer to the leading end of an existing beam while changing at least one of a beam length, the number of branches, or a rotation angle about the axis of the existing beam on the basis of a preset rule so that a plurality of beams included in the network structure of a porous structure are generated in the design space acquired by an acquirer (151); and a node joiner (153) that selects a plurality of nodes that is not directly connected to each other through a beam, among the plurality of nodes generated by the beam generator (152), to join the selected nodes to generate a single node on the basis of a preset rule.

### TECHNICAL FIELD

The present disclosure relates to a design device, a design method, a program, a porous structure, and a method of manufacturing a porous structure.

### BACKGROUND ART

In recent years, porous structures including many pores have attracted attention in fields requiring high functionality. Examples of the fields include biomedical engineering and aerospace engineering. Development of design techniques for achieving porous structures that can be manufactured by additive manufacturing technologies and have desired characteristics has been pursued. For example, Patent Literature 1 discloses a method of designing a porous structure for an implant by fusing a cell including a plurality of struts and nodes with another cell.

### CITATION LIST

### Patent Literature

Patent Literature 1: Unexamined Japanese Patent Application Publication No. 2017-200630

### SUMMARY OF INVENTION

### Technical Problem

In the design method of Patent Literature 1, characteristics such as the rigidity, orientation, and branch of each strut are not controlled. Therefore, the structural characteristics of a porous structure cannot be controlled, and the porous structure of which the mechanical characteristics depend on a load direction is obtained. In a case in which such a porous structure is applied to, for example, an implant, a sudden load from an unexpected direction may cause the porous structure to be broken. Such a problem is not limited to the case in which the porous structure is applied to an implant, but also comes up in a case in which such a porous structure is applied to another object.

The present disclosure was based on such a background. An objective of the present disclosure is to provide: a design device, a design method, and a program that enable structural characteristics and mechanical characteristics in a porous structure to be easily controlled; the porous structure of which the structural characteristics and mechanical characteristics are controlled; and a method of manufacturing the porous structure.

### Solution to Problem

In order to achieve the objective described above, a design device according to a first aspect of the present disclosure includes:
an acquirer that acquires a design space in which a porous structure model is generated;
a beam generator that repeatedly generates a plurality of new beams branching from a node closer to a leading end of an existing beam while changing at least one of a beam length, a number of branches, or a rotation angle about an axis of the existing beam on a basis of a preset rule so that a plurality of beams included in a network structure of a porous structure are generated in the design space acquired by the acquirer;
a node joiner that selects a plurality of nodes that is not directly connected to each other through a beam, among the plurality of nodes generated by the beam generator, to join the selected nodes to generate a single node on a basis of a preset rule; and
a model generator that allows each beam included in a network structure obtained by the node joiner to be a volume to generate a porous structure model.

The beam generator may generate a plurality of new beams so that each beam branching from an identical node three-dimensionally isotropically extends according to a number of branches, the number being set at each node of a beam.

In a case of generating a plurality of new beams branching from a node closer to a leading end of an existing beam, the beam generator may end generation of a beam at the node.

In a case in which a node closer to a leading end of a new beam is present outside a design space, the beam generator ends generation of a beam at the node, and
the design device may further include a beam eliminator that eliminates a beam outside the design space, among beams that are generated by the beam generator and extend outside the design space.

In a case in which a node that satisfies a preset joinability condition is present within a search range from a node closer to a leading end of a new beam, the beam generator ends generation of a beam at the node closer to the leading end of the new beam, and
the node joiner may join a node at which generation of a beam is ended due to presence of a node that satisfies the joinability condition within the search range by the beam generator to a joinable node that is closest to the node.

The beam generator sets nodes at node numbers in order of generation of the nodes, and
the node joiner may join nodes at which generation of beams is ended due to presence of a node that satisfies a joinability condition within a search range in the beam generator to joinable nodes that are closest to the nodes in increasing order of the set node numbers.

In a case in which a node density that is a number of nodes per unit volume within a search range set at a node closer to a leading end of a new beam is more than a node density threshold value, the beam generator may end generation of a beam at the node.

The model generator may transform each beam into a beam-like member having an identical cross-sectional shape in at least an intermediate portion.

In order to achieve the objective described above, a design method according to a second aspect of the present disclosure is a design method that is executed by a design device, the design method including:
acquiring a design space in which a porous structure model is generated;
repeatedly generating a plurality of new beams branching from a node closer to a leading end of an existing beam while changing at least one of a beam length, a number of branches, or a rotation angle about an axis of the existing beam on a basis of a preset rule so that a plurality of beams included in a network structure of a porous structure are generated in the acquired design space;
selecting a plurality of nodes that is not directly connected to each other through a beam, among the generated plurality of nodes, to join the selected nodes to generate a single node on a basis of a preset rule; and
allowing each beam included in a network structure obtained to be a volume to generate a porous structure model.

In order to achieve the objective described above, a program according to a third aspect of the present disclosure allows a computer to function as:
an acquiring means that acquires a design space in which a porous structure model is generated;
a beam generation means that repeatedly generates a plurality of new beams branching from a node closer to a leading end of an existing beam while changing at least one of a beam length, a number of branches, or a rotation angle about an axis of the existing beam on a basis of a preset rule so that a plurality of beams included in a network structure of a porous structure are generated in the design space acquired by the acquiring means;
a node joining means that selects a plurality of nodes that is not directly connected to each other through a beam, among the plurality of nodes generated by the beam generation means, to join the selected nodes to generate a single node on a basis of a preset rule; and
a model generation means that allows each beam included in a network structure obtained by the node joining means to be a volume to generate a porous structure model.

In order to achieve the objective described above, a porous structure according to a fourth aspect of the present disclosure is a porous structure including a plurality of beam-like members that is connected to each other through a node, wherein
each beam-like member is arranged so that an identical unit structure is prevented from being repeated in the porous structure, and
lengths of the beam-like members and a number of the beam-like members branching from the node are set to be distributed in ranges between upper and lower limits that are set, respectively.

At least one of a plurality of nodes included in the porous structure may be nodes that are arranged so that each beam-like member three-dimensionally isotropically extends from an identical node.

In order to achieve the objective described above, a method according to a fifth aspect of the present disclosure is a method of manufacturing a porous structure, the method including:
a step of manufacturing a porous structure on a basis of the porous structure model that is generated by the design device or the design method.

### Advantageous Effects of Invention

In accordance with the present disclosure, there can be provided: a design device, a design method, and a program that enable structural characteristics and mechanical characteristics in a porous structure to be easily controlled; the porous structure of which the structural characteristics and mechanical characteristics are controlled; and a method of manufacturing the porous structure.

### BRIEF DESCRIPTION OF DRAWINGS

FIG. 1 is a schematic view illustrating the configuration of a system for manufacturing a porous structure according to an embodiment of the present disclosure;

FIG. 2 is a view illustrating an example of beams that are generated by a design device according to the embodiment of the present disclosure;

FIG. 3A is a perspective view illustrating a network structure including beams generated by the design device according to the embodiment of the present disclosure;

FIG. 3B is a view illustrating a state in which the beams in FIG. 3A are allowed to be a volume;

FIG. 4 is a block diagram illustrating the hardware configuration of the design device according to the embodiment of the present disclosure;

FIG. 5A is a view illustrating an example of the data table of a parameter storage according to the embodiment of the present disclosure;

FIG. 5B is a view illustrating an example of the data table of a probability distribution storage according to the embodiment of the present disclosure;

FIG. 6A is a graph illustrating an example of the probability density function of a beam length;

FIG. 6B is a graph illustrating an example of the probability mass function of the number of branches;

FIG. 7 is a view illustrating a specific example of a beam arrangement having three-dimensional isotropy;

FIG. 8 is a view illustrating a procedure of joining two nodes to generate a single node by a design device according to an embodiment of the present disclosure;

FIG. 9 is a flow chart illustrating the flow of a design process according to an embodiment of the present disclosure;

FIG. 10 is a flow chart illustrating the flow of a beam generation process according to an embodiment of the present disclosure;

FIG. 11 is a view illustrating a procedure of joining two nodes to generate a single node by a design device according to an alternative example of the present disclosure;

FIG. 12 is a view illustrating an example of the orientation angle of a beam generated by a design device according to an alternative example of the present disclosure;

FIG. 13 is a front view illustrating a state in which a porous structure according to an alternative example of the present disclosure is deformed by a compressive load;

FIG. 14A is a view obtained by photographing the appearance of the test piece of a porous structure of the present disclosure in Examples;

FIG. 14B is a view obtained by photographing the appearance of the test piece of a diamond lattice structure in Examples; and

FIG. 15 is a graph illustrating the stress-strain relation of each test piece in Examples.

### DESCRIPTION OF EMBODIMENTS

A design device, a design method, a program, a porous structure, and a method of manufacturing a porous structure according to an embodiment of the present disclosure are described in detail below with reference to the drawings. In each drawing, the same or similar portions are denoted by the same reference characters.

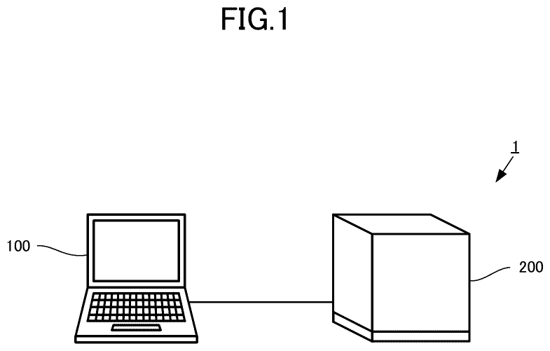

FIG. 1 is a schematic view illustrating the configuration of a system 1 for manufacturing a porous structure according to an embodiment. The manufacturing system 1 is a system that generates the model of a porous structure and manufactures the porous structure on the basis of the generated model. The manufacturing system 1 includes a design device 100 and a manufacturing device 200. The design device 100 and the manufacturing device 200 are communicatably connected to each other through a wired or wireless communication line.

The design device 100 generates a porous structure model including many beam-like members that are arranged in a three-dimensional network form in an optional design space. The porous structure model is obtained by allowing many beams included in a network structure to be a volume, and may be any of a solid model and a surface model. The network structure includes the many beams arranged in a three-dimensional network form, the beams are straight lines having no volume, and both ends of each beam have nodes.

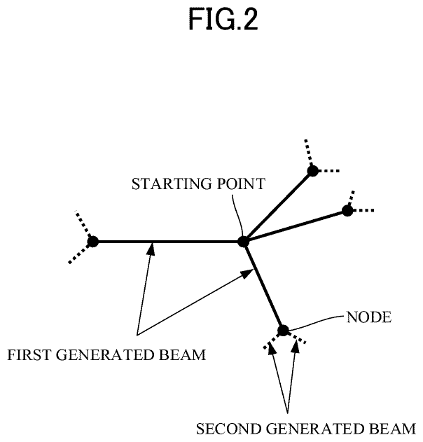

FIG. 2 is a view illustrating an example of beams that are generated by the design device 100 according to the embodiment. FIG. 2 illustrates a state in which a three-dimensional space is viewed from directly above. In FIG. 2, each beam is distributed on the three-dimensional space. Each beam is represented by a continuous or dotted line, and each node including a starting point is represented by “symbol ●” Generation of a new beam is started from a starting point set in a design space. As illustrated by the dotted lines in FIG. 2, next new beams are generated at each of nodes closer to the leading ends of four existing cantilevered beams that are first generated. New beams are generated so that all the beams belonging to an identical node three-dimensionally isotropically extend when any beams are generated. The network structure including many beams gradually grows due to repeated generation of new beams branching from nodes closer to the leading ends of existing cantilevered beams.

To randomly orient each beam of the network structure, each parameter of a beam length, the number of branches, and a rotation angle is randomly set based on a preset rule, for example, a probability distribution. Specifically, the beam length is randomly set on a beam basis, and the number of branches and the rotation angle are randomly set on a node basis. The beam length is a length between nodes at both ends of the beam. The number of branches is the number of beams branching from one node, and includes not only the number of new beams but also the number of existing beams. In the example in FIG. 2, the number of branches at the starting point is four, and each of the numbers of branches at the other nodes is three. The rotation angle is a rotation angle in a case in which a new beam is rotated about an axis extending in the longitudinal direction of an existing beam.

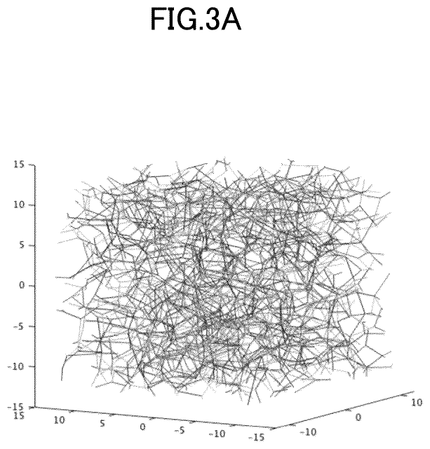

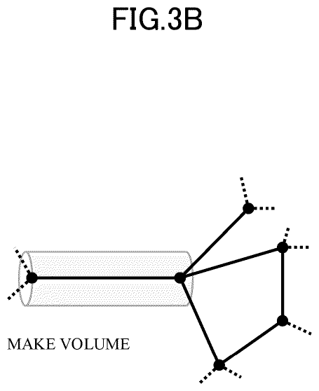

FIG. 3A is a perspective view illustrating a network structure including beams generated by the design device 100 according to the embodiment, and FIG. 3B is a view illustrating a state in which the beams in FIG. 3A are allowed to be a volume. In FIG. 3A, a network structure is generated in a design space of 25 mm×25 mm×25 mm, as an example. For example, when generation of a new beam is started from a starting point (0, 0, 0), and generation of a new beam is then repeated from a node closer to the leading end of the existing cantilevered beam that has been generated, many beams are generated so as to expand in the whole design space, as illustrated in FIG. 3A. When the beams are then allowed to be a volume so that each beam has a volume as illustrated in FIG. 3B, a porous structure model is generated. For example, each beam may be transformed into a beam-like member having an identical cross section in at least an intermediate portion in the case of allowing the beams to be the volume.

Referring back to FIG. 1, the manufacturing device 200 is a device that manufactures a porous structure on the basis of the computer aided design (CAD) data of the model generated by the design device 100. The manufacturing device 200 is, for example, an additive manufacturing machine. For example, a laser powder bed melting method in which a series of steps of making a thin metal powder layer on a base plate and selectively melting the metal powder layer by laser light is repeated is used in the additive manufacturing machine.

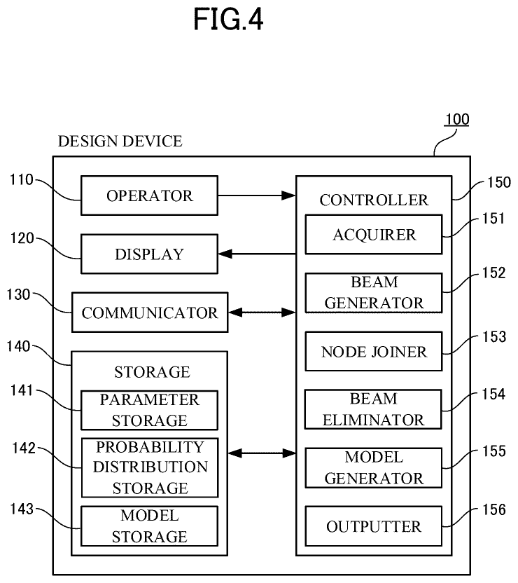

FIG. 4 is a block diagram illustrating the hardware configuration of the design device 100 according to the embodiment. For example, the design device 100 is a general purpose computer. The design device 100 includes an operator 110, a display 120, a communicator 130, a storage 140, and a controller 150. Such portions of the design device 100 are connected to each other through internal buses (not illustrated). A description is provided below by taking, as an example, a case in which a beam length is randomly changed on a beam generation basis, the number of branches and a rotation angle are randomly changed on a node basis, and each beam is transformed into a cylindrical object having a constant diameter when beams are allowed to be a volume in the design device 100.

The operator 110 accepts instructions from a user, and feeds an operation signal corresponding to the accepted operation to the controller 150. The operator 110 includes an input device such as, for example, a mouse or a keyboard.

The display 120 includes a display driving circuit, and displays various images for a user on the basis of data that is fed from the controller 150. The display 120 includes a display device such as, for example, a liquid crystal display. The display 120 displays, for example, the porous structure model generated by the design device 100.

The communicator 130 is a communication interface for communication of the design device 100 with an external instrument. The communicator 130 communicates with an external instrument through, for example, a communication network such as the Internet, or an input-output terminal such as a universal serial bus (USB).

The storage 140 includes, for example, a random access memory (RAM), a read only memory (ROM), a flash memory, and a hard disk. The storage 140 stores a program that is executed by the controller 150, and various items of data. The storage 140 also functions as a working memory for temporarily storing various items of information and allowing the controller 150 to execute a process. The storage 140 further includes a parameter storage 141, a probability distribution storage 142, a model storage 143.

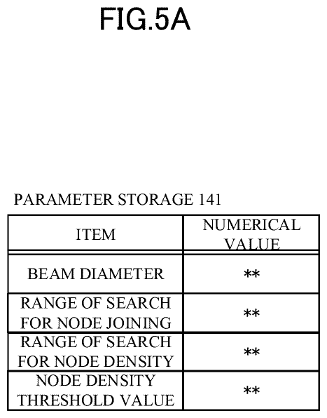

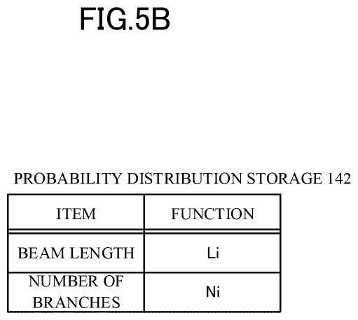

FIG. 5A is a view illustrating an example of the data table of the parameter storage 141 according to the embodiment. The parameter storage 141 stores the beam diameter of the network structure indicating the porous structure, the range of search for node joining, the range of search for a node density, and a node density threshold value. The beam diameter is the diameter of a cylindrical object in a case in which beams are allowed to be a volume and transformed into the cylindrical object, and is restricted in a range in which additive manufacturing is possible. Each of the range of search for node joining, the range of search for a node density, and a node density threshold value is a parameter relating to a condition for ending generation of a new beam at a node closer to the leading end of the new beam, and is specifically described below.

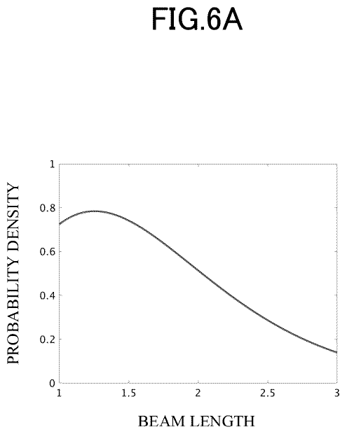

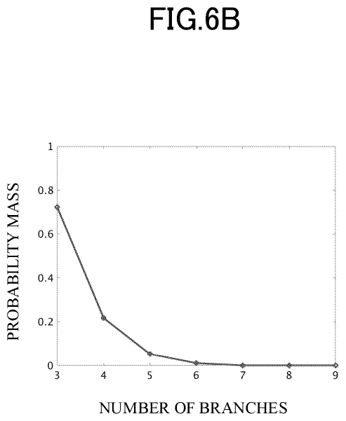

FIG. 5B is a view illustrating an example of the data table of the probability distribution storage 142 according to the embodiment. The probability distribution storage 142 stores the probability density function of a beam length and the probability mass function of the number of branches. The upper and lower limit values of each of the beam length and the number of branches are preferably set. As an example, the probability density function of a beam length is represented by a gamma probability distribution in which the upper and lower limit values of the beam length are set, as illustrated in FIG. 6A, and the probability mass distribution of the number of branches is represented by a Poisson probability distribution in which the number of branches is discretely distributed in a range of 3 to 6, as illustrated in FIG. 6B. The probability density function of a beam length is set in consideration of beam diameters stored in the parameter storage 141.

The volume density of a porous structure is one of design indices that greatly influence the mechanical characteristic of the porous structure. For example, at least one of an increase in beam diameter, a decrease in average beam length, and an increase in the average number of branches may be performed in order to increase the volume density. The node density threshold value may be increased. For example, the rigidity of the porous structure can be enhanced by increasing the volume density.

Referring back to FIG. 4, the model storage 143 stores CAD data illustrating the boundary surface of a design space set by a user and CAD data illustrating the porous structure model generated by the design device 100. For example, the boundary surface of the design space may be generated by a user by using three-dimensional CAD, and the model storage 143 may acquire the CAD data of the boundary surface generated by the three-dimensional CAD.

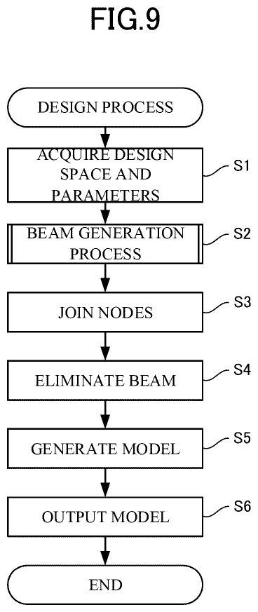

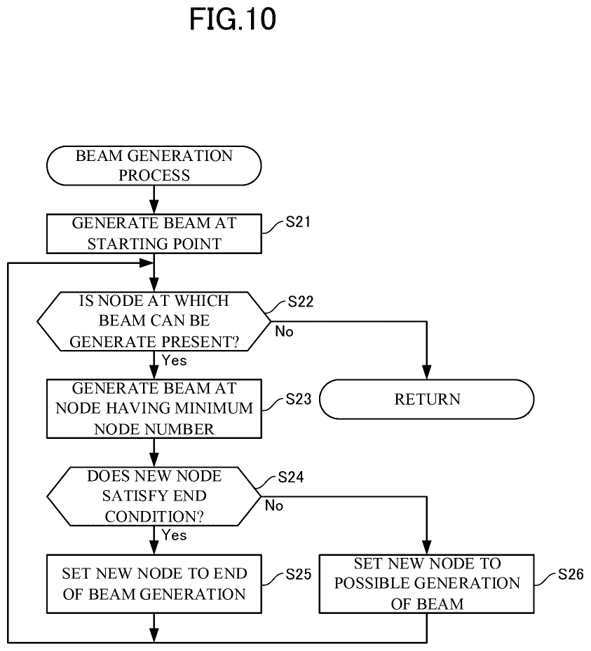

The controller 150 includes a processor, and controls each portion of the design device 100. The processor is, for example, a central processing unit (CPU). The controller 150 executes each of a design process in FIG. 9 and a beam generation process in FIG. 10 by executing a program stored in the storage 140. The controller 150 functionally includes an acquirer 151, a beam generator 152, a node joiner 153, a beam eliminator 154, a model generator 155, and an outputter 156.

The acquirer 151 acquires CAD data illustrating the boundary surface of a design space set by a user, a beam diameter, the range of search for node joining, the range of search for a node density, a node density threshold value, the probability density function of a beam length, and the probability mass function of the number of branches, and allows each thereof to be stored in any of the parameter storage 141, the probability distribution storage 142, and the model storage 143. Acquisition of data by the acquirer 151 also includes readout of data stored in the storage 140.

The beam generator 152 repeatedly generates a plurality of new beams branching from nodes closer to the leading end of an existing beam so that many beams included in the network structure of the porous structure are generated in the design space acquired by the acquirer 151. Specifically, generation of beams is started at a starting point set in the design space as illustrated in FIG. 2, and generation of new beams branching from nodes closer to the leading ends of the existing cantilevered beams that have been generated is then repeated.

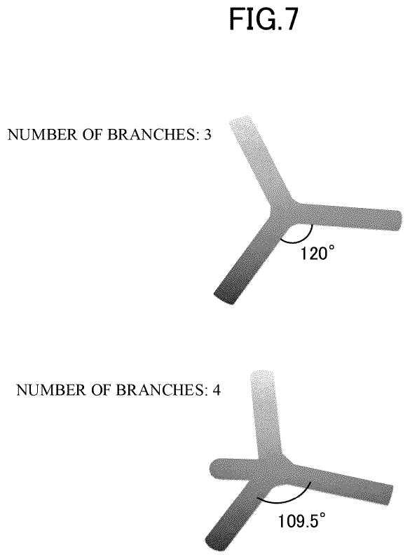

Each beam branching from an identical node is generated to extend in a three-dimensionally isotropic manner depending on the assigned number of branches. For example, when the number of branches is three, a branching angle that is an angle made by beams adjacent to each other, among a plurality of beams belonging to an identical node, may be set at 120°, as illustrated in the upper section of FIG. 7. When the number of branches is four, a branching angle may be set at 109.5° because each beam is arranged in a tetrapod block shape as illustrated in the lower section of FIG. 7.

Based on a preset rule, the beam generator 152 randomly changes a beam length on the basis of generation of a beam at a node closer to the leading end of an existing beam, and the number of branches and a rotation angle on a node basis. The beam length and the number of branches are changed based on the probability density function and the probability mass function stored in the probability distribution storage 142 in FIG. 5B, and the rotation angle is changed at an equal probability. In order to randomly change the beam length, the number of branches, and the rotation angle, a random number may be generated according to a rule set for each of the beam length, the number of branches, and the rotation angle. Such a random number encompasses a pseudorandom number. By randomly changing the beam length, the number of branches, and the rotation angle on the basis of generation of a beam, the beam length and orientation of each beam are randomly distributed, and structural isotropy in the network structure is obtained in macroscopic view.

The beam generator 152 sets a node number i at each node in order on the basis of generation of a beam. Node numbers i different from each other are set in order at nodes closer to the leading ends of a plurality of new beams branching from an identical node. For example, when the is number of branches is four, individual node numbers i are set at newly generated three nodes, respectively. Such node numbers i are used for defining the order of joining nodes in a node joining process described later.

The beam generator 152 determines whether a new beam can be generated or generation of a new beam is ended on a node basis. The beam generator 152 ends generation of a new beam at a node in a case in which the conditions of end of generation of a new beam are satisfied. Generation of a new beam at each node is ended in the case of satisfying any of the following four end conditions (1) to (4). Hereinafter, a node closer to the leading end of a new beam is referred to as “new node” in order to facilitate understanding.

### End Condition (1)

When a plurality of new beams branching from a node closer to the leading end of an existing beam is generated, generation of a beam at the node is ended.

### End Condition (2)

When a new node is present outside a design space, generation of a beam at the new node is ended. Setting of the end condition (2) results in generation of a network structure that slightly extends off the boundary surface of a design space after end of repeated generation of a beam. A beam having a node at which generation of a beam is ended on the end condition (2) extends outside a design space, and therefore, a portion extending outside the design space is eliminated in a process described later.

### End Condition (3)

Generation of a beam at the new node is ended when a new node does not satisfy the end condition (2), and a node that satisfies a preset joinability condition is present in a search range from the new node. A new node that satisfies the end condition (3) is joined to a joinable node in a process described later. The search range is, for example, a spherical region of which the center is a new node, and is represented by the radius of the spherical region. The node that satisfies the joinability condition in generation of a beam is only a node that satisfies the end condition (1) or (2) at the time of determining whether a new node satisfies the end condition (3), and does not include nodes at both ends of an existing beam from which a new beam to which the new node belongs branches.

### End Condition (4)

When a new node satisfies neither the end condition (2) nor (3), and a node density in a search range from the new node is more than a node density threshold value, generation of a beam at the new node is ended. The search range is, for example, a spherical region of which the center is a new node, and is represented by the radius of the spherical region. The node density is the number of nodes that are present in a search range and enable generation of a new beam at the time of determining whether the new node satisfies the end condition (4) or nodes that satisfies the end condition (1) or (2) per unit volume. The end condition (4) is set because beams cluster close together in a periphery region when further generation of a beam is repeated at a new node that satisfies the end condition (4). The end condition (4) may be omitted if the range of search for node joining and the probability distributions of a beam length and the number of branches are set appropriately.

The end conditions of generation of a beam are described above.

The node joiner 153 joins a node at which generation of a new beam is ended due to the presence of a node that satisfies a joinability condition in a search range in the beam generator 152 to a joinable node closest to the node. A joinable node at which a node at which generation of a new beam is ended is joined by the node joiner 153 does not necessarily the same as a joinable node considered to be the closest to the new node at the time of determining that a node that satisfies a joinability condition is present. This is because, for example, a node that newly satisfies a joinability condition may be generated in a closer area after determination that a node that satisfies the joinability condition is present.

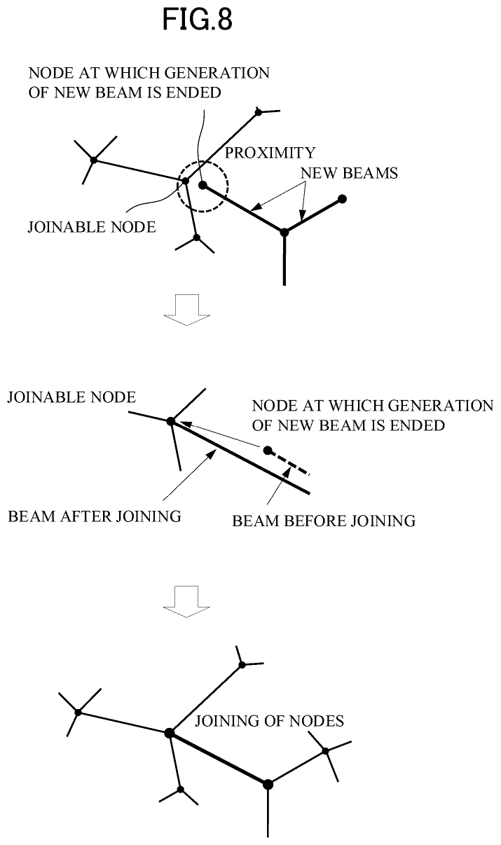

A process of joining individual nodes is described with reference to a specific example in FIG. 8. First, for a node at which generation of a new beam is ended due to the presence of a node that satisfies a joinability condition in a search range in the beam generator 152, a joinable node closest to the node is searched. A node at which generation of a new beam is ended due to the presence of a node that satisfies the joinability condition in the search range in the beam generator 152 as illustrated in a region circled in a dashed line in FIG. 8 is joined to a joinable node closest to the node. Specifically, a node at which generation of a new beam is ended is moved to overlap with a joinable node closest to the node, and the gradient and length of a beam to which the node belongs are also varied with the movement of the node.

Nodes are joined in the following procedure after end of repeated generation of beams by the beam generator 152. For nodes at which generation of new beams is ended due to the presence of nodes that satisfy a joinability condition in a search range in the beam generator 152, nodes joinable at a node having a node number i are searched in increasing order of set node number i, and a joinable node at the closest position among the joinable nodes and a node having a node number i are joined to generate a single node. In such a case, the joinable nodes are regarded as nodes satisfying the end condition (1) or (2) at the time of end of repeated generation of a beam by the beam generator 152, and includes neither a beam to which a node having a node number i belongs nor a node belonging to a beam that is directly connected to the beam. When a plurality of joinable nodes at an equal distance is present, the node having the lowest node number i is selected and joined.

The joining of nodes can be minimized by performing the joining process in the procedure described above. As a result, structural isotropy in the network structure can be mostly maintained.

In the network structure obtained by the node joiner 153, the beam eliminator 154 eliminates part of beams present outside the design space and all of cantilevered beams present in an interior that does not come into contact with the boundary surface of the design space. Specifically, the beam eliminator 154 eliminates portions of the cantilevered beams of which some satisfying the end condition (2) for beam generation in the beam generator 152 are outside the design space acquired by the acquirer 151, the portions being outside the design space. The beam eliminator 154 eliminates cantilevered beams in the interior of the network structure obtained by the node joiner 153. The reason that such a cantilevered beam is eliminated is because the beam does not contribute to transmission of a load for the structure.

The model generator 155 allows each beam of the network structure in which some beams included in the network structure are eliminated by the beam eliminator 154 to be a volume, to generate a porous structure model. Specifically, all the beams included in the network structure are transformed into a cylindrical object having a beam diameter acquired by the acquirer 151, and a sphere having a diameter that is the beam diameter is also set at each node, whereby CAD data illustrating the porous structure model is generated. Surface quality at each node can be improved by applying the sphere to each node in the case of generating the volume.

The outputter 156 outputs the porous structure model generated by the model generator 155. The outputter 156 displays, for example, the CAD data of the porous structure model generated by the model generator 155 on the display 120. The outputter 156 controls, for example, the communicator 130 to transmit the CAD data of the porous structure model generated by the model generator 155 to the manufacturing device 200.

The hardware configuration of the design device 100 is described above.

### (Design Process)

Next, a design process that is executed by the controller 150 of the design device 100 according to the embodiment is described with reference to FIG. 9. The design process is a process of generating the CAD data of a porous structure model in a design space specified by a user. The design process is started when the user launches the application of the design device 100.

The design device 100 requests a user to provide instructions for various parameters and CAD data illustrating the boundary surface of a design space. Such various parameters include a beam diameter, the range of search for node joining, the range of search for node density, a node density threshold value, a beam length, and the number of branches. Depending on the request, the user sets the various parameters and the CAD data illustrating the boundary surface of the design space. In such a case, a beam length is set based on a probability density function, and the number of branches is set based on a probability mass function. The acquirer 151 acquires the various parameters and the CAD data illustrating the boundary surface of the design space set by the user, and allows each thereof to be stored in any of the parameter storage 141 in FIG. 5A, the probability distribution storage 142 in FIG. 5B, and the model storage 143 (step S1).

Then, the beam generator 152 executes a beam generation process in which generation of a beam is repeated until beams expand to the whole design space acquired by the process of step S1 (step S2). The flow of the beam generation process that is executed by the beam generator 152 is described below with reference to FIG. 10.

### (Beam Generation Process)

First, the beam generator 152 randomly sets a beam length, the number of branches, and a rotation angle, and performs the first beam generation at a starting point on the basis of the set beam length, number of branches, and rotation angle (step S21). The starting point is preset in a design space by a user. The beam length and the number of branches are set based on the probability density function of a beam length and the probability mass function of the number of branches, the probability density function and the probability mass function being stored in the probability distribution storage 142 in FIG. 5B. The rotation angle is randomly set at an equal probability. Each beam belonging to an identical node is generated to three-dimensionally isotropically extend depending on the assigned number of branches. In the example of FIG. 2, four beams are generated from the starting point. However, the number of branches from a starting point may be other than four. After the generation of the beams, the starting point is set to “end of beam generation”, and a new node is set to “possible generation of beam”. Subsequently, a node number i is set in order at a new node when a beam is generated at each node. The node number at the starting point is i=1. Node numbers i that are different from each other are set in order at nodes closer to the leading ends of a plurality of new beams generated from an identical node.

Then, the beam generator 152 determines whether a node at which a new beam can be generated is present (step S22). When determining that a node at which a new beam can be generated is present (step S22; Yes), the beam generator 152 randomly sets a beam length, the number of branches, and a rotation angle, and generates a beam at a node, of which the node number is minimum and at which a beam can be generated, on the basis of the set beam length, number of branches, and rotation angle (step S23). In such a case, the beam length is randomly set on a beam basis, and the number of branches and the rotation angle are randomly set on a node basis. After the generation of the beam, the node is set at “end of beam generation”. In contrast, when it is determined that a beam node at which a new beam can be generated is not present (step S22; No), the process is returned.

After the process of step S23, the beam generator 152 determines whether a new node generated by the process of step S23 satisfies a condition for ending generation of a new beam at the node (step S24). Specifically, the condition for ending generation of a new beam at each node may be any of a case in which a plurality of new beams branching from a node closer to the leading end side of an existing beam is generated, a case in which a new node is present outside a design space, a case in which a node satisfying a joinability condition in a search range from a new node is present, and a case in which a node density in the range of search for a new node is more than a node density threshold value. As the range of search for node joining, the range of search for a node density, and the node density threshold value, the range of search for node joining, the range of search for a node density, and a node density threshold value, stored in the parameter storage 141 in FIG. 5A, may be read, respectively.

When determining that the new node satisfies the condition for ending generation of a new beam (step S24; Yes), the beam generator 152 sets the new node at “end of beam generation” (step S25), and the process is returned to step S22. In contrast, when determining that the new node does not satisfy the condition for ending generation of a new beam (step S24; No), the beam generator 152 sets the new node at “possible generation of beam” (step S26), and the process is returned to step S22.

The flow of the process of generating a beam is described above.

After return to the design process in FIG. 9, the node joiner 153 joins a node at which generation of a new beam is ended due to the presence of a node satisfying a joinability condition in a search range in the process of step S2 to a joinable node closest to the node (step S3). Specifically, for nodes in the case of determining that the nodes satisfying the joinability condition are present in the search range in the process of step S2, joinable nodes in nodes having node numbers i are searched in increasing order of node number i, and a joinable node that is at the closest position among the joinable nodes and a node having a node number i are joined to generate a single node. When a plurality of joinable nodes at an equal distance is present, a node having the lowest node number i may be selected.

Then, the beam eliminator 154 eliminates part of beams present outside a design space and all of cantilevered beams present in an interior that does not come into contact with the boundary surface of the design space in the network structure obtained in the process of step S3 (step S4).

Then, the model generator 155 allows each beam of the network structure obtained in the process of step S4 to be a volume to generate the CAD data of the porous structure model, and allows the CAD data to be stored in the model storage 143 (step S5). In the generation of the volume, each beam is transformed into a cylindrical object of which the diameter is a beam diameter stored in the parameter storage 141 in FIG. 5A, and a sphere of which the diameter is the beam diameter is set at each node.

Then, the outputter 156 outputs the porous structure model generated in the process of step S5 to the outside (step S6), and the process is ended. For example, in a case in which a user instructs the model to be displayed, the outputter 156 may allow the porous structure model to be displayed on the display 120. In a case in which a user instructs an actual porous structure to be manufactured by the manufacturing device 200, the outputter 156 may transmits the CAD data of the porous structure to the manufacturing device 200 through the communicator 130.

The flow of the design process is described above.

When acquiring the CAD data of the porous structure from the design device 100, the manufacturing device 200 executes slicing for determining a tool path on the basis of the CAD data. The tool path is a path through which a laser beam from the manufacturing device 200 is moved. The manufacturing device 200 executes additive manufacturing on the basis of the result of the slicing to obtain the actual porous structure based on the porous structure model. The porous structure obtained in the step described above is not limited to a porous structure that strictly reflects the shape of the porous structure model, but the influence of dimensional accuracy realizable in additive manufacturing is tolerated. For example, a portion to which a plurality of cylindrical objects is connected in the porous structure may be rounded.

The porous structure obtained by the manufacturing device 200 according to the embodiment is a porous structure including a plurality of bar-like members connected to each other between nodes, and each bar-like member is arranged so that repetition of an identical unit structure does not occur in the porous structure. The bar-like member is an example of a beam-like member through which nodes are connected to each other. Each of the length of the bar-like member and the number of bar-like members branching from the node is set to be distributed in a set range between upper and lower limits, and arranged so that the directions of extension of the bar-like members are not limited to a specific direction. The porous structure is preferably formulated so that the arrangement of each bar-like member does not correspond to the arrangement of Voronoi sides formulated by the Voronoi tessellation of a design space. The porous structure may include an identical shape in at least the intermediate portion of each bar-like member.

The porous structure includes many nodes, and some of the many nodes are nodes that are arranged so that each bar-like member three-dimensionally isotropically extends from an identical node. The three-dimensionally isotropic extension of each bar-like member means that N bar-like members extending from an identical node are arranged along the direction of a unit vector linking an origin point and each point to each other in a case in which N points are equally or equivalently arranged on a unit sphere of which the center is the origin point. The equal arrangement of N points is, for example, such an arrangement that the minimum value of a spherical distance between the points is maximum, among possible arrangements of the N points. For example, the arrangement of each bar-like member in a case in which N points are equally arranged at N=3, 4 corresponds to the beam arrangement illustrated in the specific example in FIG. 7. For example, equivalence to an identical node includes a case in which the absolute value of a difference between each angle between bar-like members and an angle in a case in which point groups are equally arranged on a unit sphere is, for example, 10° or less, preferably 5° or less.

The porous structure includes the configuration described above, and therefore has the following advantages in comparison with a regular lattice structure (for example, diamond lattice structure) including repetitions of a unit structure. First, the porous structure according to the embodiment does not have structural anisotropy such as the structural anisotropy of a regular lattice structure, as a result, is also mechanically isotropic, and can therefore endure a load even in a sudden, unexpected direction. The porous structure according to the embodiment does not have an incomplete unit structure on the surface of a design space, such as the incomplete unit structure of a regular lattice structure, and can therefore prevent local fracture on the surface of the design space. In addition, in the porous structure according to the embodiment, a decrease and variation in stress after initial maximum compression stress in compression loading indicated by a regular lattice structure are suppressed, and therefore, design stress can be highly set, and growth of fracture can also be suppressed. Accordingly, the porous structure is suitable for application to technical fields, in which fatal growth of fracture is not permitted, such as biomedical engineering and aerospace engineering.

As described above, the design device 100 according to the embodiment includes: the beam generator 152 that repeatedly generates a plurality of new beams branching from a node closer to the leading end of an existing beam while changing at least one of a beam length, the number of branches, and a rotation angle about the axis of the existing beam on the basis of a preset rule and ends generation of beams at nodes closer to the leading ends of the new beams in a case in which a node satisfying a preset joinability condition is present in a search range from the nodes closer to the leading ends of the new beams; and the node joiner 153 that selects a plurality of nodes that is not directly connected to each other through a beam, among many nodes generated by the beam generator 152, to join the selected nodes to generate a single node on the basis of a preset rule. Therefore, a porous structure model having mechanical isotropy can be easily designed in an optional design space.

The present disclosure is not limited to the embodiment described above, and an alternative example described below is also possible.

### Alternative Example

In the embodiment described above, a beam diameter, the range of search for node joining, the range of search for a node density, a node density threshold value, the probability density function of a beam length, and the probability mass function of the number of branches are set by a user. However, the present disclosure is not limited thereto. For example, the design device 100 may generate the above-described parameters and probability distribution on the basis of a condition specified by a user, or the design device 100 may acquire such parameters and probability distribution generated by an external computer.

In the embodiment described above, a beam length, the number of branches, and a rotation angle are changed on a beam generation basis. However, the present disclosure is not limited thereto. For example, any one of a beam length, the number of branches, and a rotation angle may be changed on a beam generation basis, or two of a beam length, the number of branches, and a rotation angle may be changed on a beam generation basis. The kind of a parameter that is randomly set on a beam generation basis may also be changed each time without limitation to the case of randomly setting an identical parameter at the time of all beam generations. In addition, a beam length, the number of branches, and a rotation angle may be changed on a node basis at the time of generation of a beam.

In the embodiment described above, a beam length is represented by a gamma probability distribution, and the number of branches is represented by a Poisson probability distribution. However, the present disclosure is not limited thereto. A beam length and the number of branches may be represented by, for example, a uniform probability distribution. A beam length and the number of branches are not necessarily randomly generated based on a probability distribution, but may be set based on a predetermined rule in a numerical range set on a beam generation basis.

In the embodiment described above, a new beam is generated so that an existing beam and the new beam belonging to an identical node are three-dimensionally isotropic. However, the present disclosure is not limited thereto. A new beam may be generated so that angles between beams adjacent to each other among a plurality of beams belonging to an identical node are different from each other.

In the embodiment described above, only one starting point is set in a design space. However, the present disclosure is not limited thereto. Two or more starting points may be set in a design space. In such a case, a network structure may be integrated by connecting beams belonging to the starting points to each other. For example, in a case in which two starting points are set in a design space, when a distance between a node closer to the leading end of a beam belonging to one starting point and a node closer to the leading end of a beam belonging to the other starting point is not more than a threshold value, a network structure may be integrated by joining both the nodes to generate a single node.

In the embodiment described above, a condition for ending generation of a new beam is preset, and generation of a beam is controlled based on the condition. However, the present disclosure is not limited thereto. For example, it is acceptable that the target generation number of beam generations is stored in the parameter storage 141, new beams are simultaneously repeatedly generated from all of nodes closer to the leading ends of existing cantilevered beams, and arrival of the number of repetitions at the target generation number is allowed to be the condition for ending generation of a new beam. The generation number is the number of times of generation of a beam at the same timing, and the target generation number is the targeted number of generations.

In the embodiment described above, two nodes defined on a node joining condition are joined to generate a single node after generation of a network structure including many beams. However, the present disclosure is not limited thereto. For example, two nodes defined on the condition (3) for ending generation of a new beam may be joined to a single node at the time of generation of a new beam.

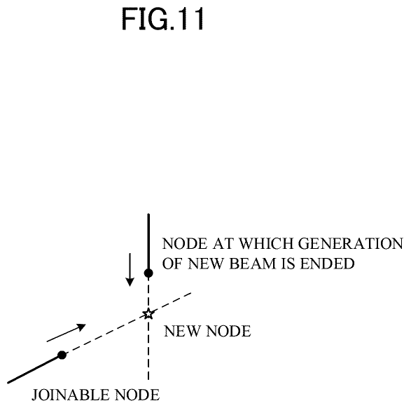

In the embodiment described above, a node at which generation of a new beam is ended is moved to overlap with a node that is closest to the node and satisfies a joinability condition as illustrated in FIG. 8. However, the present disclosure is not limited thereto. For example, a new node may be set at a point of intersection of the extended line of a beam to which a node at which generation of a new beam is ended belongs and the extended line of any beam to which a node that is closest to the node and satisfies a joinability condition belongs, as indicated by “symbol *” in FIG. 11.

In the embodiment described above, a node at which generation of a new beam is ended due to the presence of a node satisfying a joinability condition in a search range from a node closer to the leading end of a new beam in the beam generator 152 and a joinable node closest to the node are joined to generate a single node. However, the present disclosure is not limited thereto. For example, a node at which generation of a new beam is ended due to the presence of a node satisfying a joinability condition in a search range in the beam generator 152 and a joinable node that is at a position second closest to the node rather than a joinable node that is at a position closest to the node may be joined to generate a single node.

In the embodiment described above, a cantilevered beam in a structure that does not come into contact with the boundary surface of a design space is eliminated. However, the present disclosure is not limited thereto. For example, the process (step S4) of eliminating a cantilevered beam may be omitted in a case in which the volume density of a porous structure is intended to be increased, and the like.

In the embodiment described above, a cantilevered beam is present on the surface of a structure coming into contact with the boundary surface of a design space. However, the present disclosure is not limited thereto. For example, the leading ends of cantilevered beams adjacent to each other on a structure surface may be connected through an additional beam to make a network structure on the surface. A plurality of surfaces may be affixed to cover the end surface of a cantilevered beam along the boundary surface of the design space. The surfaces that are affixed to the end surface of the cantilevered beam may be planar or curved. As a result, strength can be secured even on the surface of the porous structure, and macroscopic deformation can be suppressed. The above-described technique is also useful in view of improvement in the property of manufacturing a porous structure because the conditions of the length and orientation angle of a cantilevered beam that can be manufactured by the manufacturing device can be relaxed by both-end supporting.

In the embodiment described above, a network structure is generated so as to slightly protrude from the boundary surface of a design space. However, the present disclosure is not limited thereto. For example, a sphere space containing a design space is set, a network structure is generated in the sphere space, and a portion of the network structure, outside the boundary surface of the design space, may be then eliminated.

In the embodiment described above, each beam is transformed into a cylindrical object, and spheres having the same diameter are set at nodes to allow a network structure including many beams to be a volume. However, the present disclosure is not limited thereto. For example, the cross-sectional shape of a beam-like member obtained by allowing beams to be a volume may be allowed to be a polygonal shape such as an elliptical shape, a triangular shape, a square shape, or a rectangular shape. The beam-like member is not limited to a bar-like member, and may be, for example, a plate-like member.

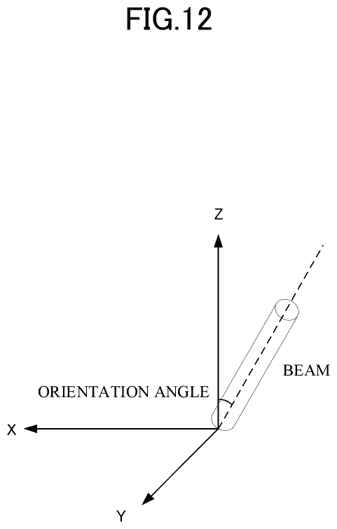

In the embodiment described above, a porous structure having structural isotropy is generated. However, the present disclosure is not limited thereto. In the model of a porous structure, structural anisotropy may be intentionally imparted by adjusting beams and branches according to an orientation angle. The orientation angle of a beam is a parameter representing the posture of the beam based on a reference coordinate system set in a design space. For example, when the reference coordinate system is an orthogonal coordinate system including the XYZ axes, the orientation angle may be represented by an angle with respect to the Z-axis as illustrated in FIG. 12.

In order to allow the model to have structural anisotropy, for example, an additional process of allowing a network structure to have structural anisotropy may be performed after end of the process (step S4) of generating a network structure having structural isotropy. In order to impart structural anisotropy, for example, the orientation directions of all the beams with respect to a reference coordinate system may be changed, and, specifically, the whole network structure may be stretched and shrunk in one direction. A beam in the preset range of an orientation angle may be eliminated, or a new beam having an orientation angle in a preset range may be added.

As another technique, a process of imparting structural anisotropy to the model may be performed when a beam is generated in the beam generation process (step S2). For example, a beam length may be increased or decreased according to an orientation angle when a beam is generated, or at least one of the number of branches and a rotation angle may be changed. Specifically, a weighting factor corresponding to an orientation angle is preset, and the weighting factor may be multiplied by any of the probability density function of a beam length and the probability mass function of the number of branches, the probability density function and the probability mass function being read from the probability distribution storage 142, and the probability density function of a rotation angle, the probability density function being stored in the storage 140. The weighting factor is, for example, a value obtained by adding 0.5 to the cosine value of an angle with respect to the Z-axis.

As an example of increasing and decreasing a beam length according to an orientation angle, a weighting factor may be set at a value obtained by adding 0.5 to the cosine value of an angle with respect to the Z-axis, and the beam length may be set at a length obtained by multiplying the weighting factor by a beam length determined by a probability density function. In such a case, the length of a beam orienting on the Z-axis is 1.5 times a beam length determined by the probability density function, and the length of a beam orienting in the XY plane is 0.5 time the beam length. As a result, structural anisotropy having a beam length distribution from the Z-axis to the X-axis and the Y-axis can be imparted to a porous structure model.

In another technique of imparting structural anisotropy to the model at the time of generating a beam, the beam length of beams of which the orientation angles that are angles with respect to the Z-axis are more than a threshold value and the beam lengths are more than a threshold value may be shortened at a certain rate or shortened to a certain beam length, and beams connected to the beams may be extended. The above-described technique can also be applied to elimination of a beam having an orientation angle and a length, the beam being difficult to manufacture by a manufacturing device, for example, in the case of layering in the Z-axis direction by additive manufacturing, and is therefore also useful in view of improvement in the property of manufacturing a porous structure.

In another technique, a process of imparting structural anisotropy to the model may be performed in the process of determining the end of generation of a new beam in the beam generation process (step S2), and the node joining process (step S3). For example, when a node satisfying a joinability condition in a search range is searched in the process of step S2, whether the orientation angle of a beam in the case of joining to each joinable node is in the preset range of an orientation angle may be determined, and generation of a new beam may be ended in the case of the presence of a joinable node in the range of the orientation angle. Then, a new node may be joined to a node closest to a new node among joinable nodes in the preset range of the orientation angle in the process of step S3.

In the porous structure obtained by the technique described above, beams are arranged in various directions in comparison with a lattice structure in which the orientation angles of beams are limited to several kinds, and therefore, structural anisotropy in which a mechanical characteristic in an orientation in which a load is applied is gently changed can be realized. The technique described above can also be applied to elimination of a beam difficult to manufacture by a manufacturing device from the model, and is useful in view of improvement in the property of manufacturing a porous structure.

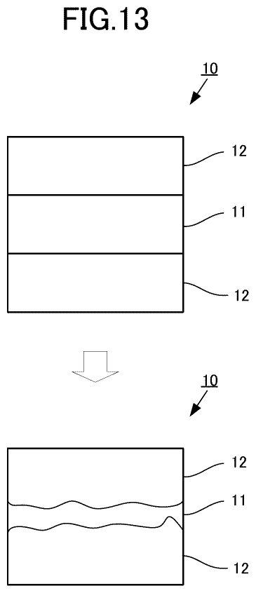

In relation to the alternative example described above, the model of a porous structure may be intentionally allowed to include an inclined structure. The inclined structure is one of structures having structural anisotropy, and is such a structure that at least one of parameters that influence structural characteristics, for example, parameters such as a beam length, the range of search for node joining, the number of branches, the range of search for a node density, a node density threshold value, and a beam diameter is distributed in the model. Disposition of the inclined structure in the porous structure enables mechanical characteristics to be changed by a region, and the behaviors of deformation and fracture to be intentionally controlled. In the inclined structure, for example, a weak region in which deformation or fracture is prone to occur and a strong region in which deformation or fracture is inhibited may be disposed in the porous structure. As an example, such a configuration that a weak region 11 is first deformed in the case of applying a compressive load in a direction in which the regions are stacked can be enabled by sandwiching the weak region 11 in a porous structure 10 between strong regions 12 in a pair, as illustrated in FIG. 13.

In order to allow the model to include an inclined structure, for example, any of a beam length, the range of search for node joining, and the number of branches may be distributed according to the coordinates in a design space in the beam generation process (step S2). In order to allow the beam length to be distributed according to the coordinates in the design space, specifically, a weighting factor depending on the coordinates in the design space may be set, and multiplied by the probability density function of the beam length read from the probability distribution storage 142. As an example of the weighting factor, the origin point of a reference coordinate system may be set at the center of the porous structure, a weighting factor at the origin point may be set at 0.5, and may be linearly changed along the Z-axis coordinate so that each of the maximum and minimum positions on the Z-axis coordinate is 1.5. In such a case, the distribution of a beam length according to the Z-axis coordinate can be applied to the porous structure model.

In another technique, each beam may be transformed into a truncated-cone-shaped bar-like member in the process (step S5) of allowing a model to be a volume, and the diameter of each bar-like member may be changed in a lengthwise direction depending on the coordinates in a design space in such a transform process. Specifically, the origin point of a reference coordinate system may be set at the center of a porous structure, a diameter that is linearly changed toward the maximum and minimum positions of the Z-axis coordinate from the origin point may be preset, and each beam may be transformed into a truncated-cone-shaped bar-like member in a case in which the diameters of nodes at both ends of each beam on the Z-axis coordinate are regarded as the diameters of the upper and lower surfaces of a truncated cone. In such a case, the distributions of beam diameters and volume densities depending on the Z-axis coordinate can be applied to the porous structure model.

In another technique, the density distribution of nodes may be changed depending on the coordinates in a design space by changing a node density threshold value that is one of conditions for ending generation of a new beam depending on the coordinates in the design space in the process of step S24 included in the beam generation process (step S2).

In the porous structure having an inclined structure, the porous structure being obtained by the technique described above, the behaviors of deformation and fracture can be controlled, an elastic modulus, an offset stress, and a plateau stress can be changed while keeping a high absorbed energy property in comparison with an isotropic porous structure having an identical or equivalent mass, and therefore, the porous structure is suitable as, for example, a shock absorption material.

In the embodiment described above, various items of data are stored in the storage 140 of the design device 100. However, the present disclosure is not limited thereto. For example, all or some of various items of data may be stored in an external control device or computer through a communication network.

In the embodiment described above, the design device 100 is operated based on the program stored in the storage 140. However, the present disclosure is not limited thereto. For example, the functional configuration realized by the program may be implemented by hardware.

In the embodiment described above, the design device 100 is a general purpose computer. However, the present disclosure is not limited thereto. For example, the design device 100 may be realized by a dedicated system, or may be realized by a computer disposed on the cloud.

In the embodiment described above, the processes executed by the design device 100 are implemented by executing the program stored in the storage 140 by the device including the physical configuration described above. However, the present disclosure may be implemented as a program, or may be implemented as a storage medium in which the program is recorded.

A device that executes the process operation described above may be configured by storing a program for executing the process operation described above in a non-transitory computer-readable recording medium such as a flexible disk, a compact disk read-only memory (CD-ROM), a digital versatile disk (DVD), or a magneto-optical disk (MO), distributing the non-transitory computer-readable recording medium, and installing the program on a computer.

In the embodiment described above, the metallic porous structure is manufactured using the laser powder bed melting method. However, the present disclosure is not limited thereto, and may be applied to manufacture of a porous structure including a ceramic or resin material. For example, a porous structures including resin may be manufactured using material extrusion (MEX). The manufactured porous structure can be used in various applications, for example, medical instruments (for example, implants), structural materials for transport apparatuses, building materials for building, and shock absorbers.

The embodiments described above are examples, the present disclosure is not limited thereto, and various embodiments are possible without departing from the gist of the invention described in claims. The components described in the embodiment and the alternative example can be freely combined. An invention equivalent to the invention described in claims are also included in the present disclosure.

The present disclosure is specifically described below with reference to Examples. However, the present disclosure is not limited to these Examples.

### EXAMPLES

In Examples, a porous structure was manufactured using a technique according to the embodiment described above, and the mechanical characteristics of the porous structure were evaluated. First, a porous structure was designed in a design space of 25 mm×25 mm×25 mm, and manufactured by additive manufacturing. Specifically, first, a network-like skeleton was generated by optionally assigning the length and number of branches of each beam included in the skeleton of the porous structure according to a probability distribution. Then, the volume of a columnar shape having a constant diameter was assigned to all the beams of the network structure, and the volume of a spherical shape of the same diameter was assigned to nodes to assign a volume to the network structure and to generate a porous structure model.

The probability density function of the beam length of each beam included in the skeleton of the porous structure was provided by a gamma probability distribution, and the upper and lower limit values of the probability density function were set. In such a case, the upper and lower limit values were provided in the range of the length of a beam that was able to be horizontally shaped by a manufacturing device for the cross-section dimension of a beam that was manufactured. The probability mass function of the number of branches was provided by a Poisson probability distribution, and the number of branches was restricted to three or four. Each of the probability density functions of a beam diameter and a beam length and the probability mass function of the number of branches were set so that the volume density of the porous structure was about 40%. The designed porous structure was confirmed to include the unbiased orientation direction of beams in macroscopic view and have no structural anisotropy.

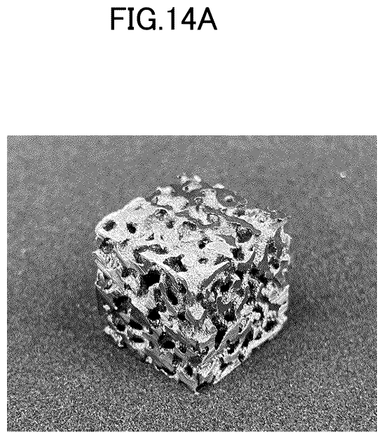

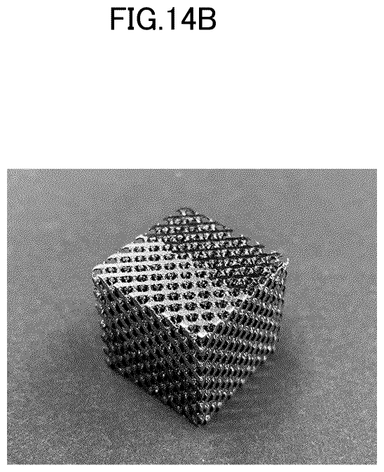

Then, a porous structure was manufactured using a laser powder bed melting method by an additive manufacturing device (LUMEX Avance-25 manufactured by Matsuura Machinery Corporation). A maraging steel powder (Matsuura Maraging II manufactured by Matsuura Machinery Corporation) was used as a material. After the additive manufacturing, the six surfaces of a sample were machined, and a test piece of 20 mm×20 mm×20 mm illustrated in FIG. 14A was produced. For a comparative purpose, a test piece having a standard diamond lattice structure having the equal dimension and the equal volume density, illustrated in FIG. 14B, was produced.

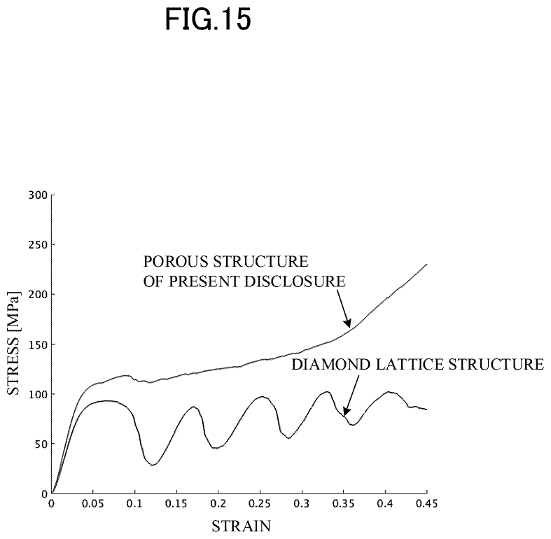

Both were subjected to a compression failure test according to ISO13314. In the compression failure test, a mechanical testing machine (AG-250kND manufactured by SHIMADZU CORPORATION) was used. The results are illustrated in FIG. 15. In the macroscopic stress-strain relation of the diamond lattice structure, the first maximum compressive stress was illustrated, the stress was then greatly decreased, and fracture was grown with laminar fracture while repeating large undulated variations. In contrast, in the porous structure of the present disclosure, a decrease and variation in stress after initial maximum compression stress were confirmed to be suppressed to greatly improve absorbed energy. Therefore, the porous structure of the present disclosure can be understood to enable suppression of the growth of fracture in compression loading, whereby a design stress can be set at a high level, and absorbed energy can be improved.

This application claims the benefit of Japanese Patent Application No. 2022-58004, filed on Mar. 31, 2022, the entire disclosure of which is incorporated by reference herein.

### INDUSTRIAL APPLICABILITY

The design device, the design method, and the program of the present disclosure enable structural characteristics and mechanical characteristics in a porous structure to be easily controlled, and structural characteristics and mechanical characteristics are controlled in the porous structure and the method of manufacturing the porous structure of the present disclosure. Therefore, the design device, the design method, the program, the porous structure, and the method of manufacturing the porous structure are useful.

### REFERENCE SIGNS LIST

1 Manufacturing system
10 Porous structure
11 Weak region
12 Strong region
100 Design device
110 Operator
120 Display
130 Communicator
140 Storage
141 Parameter storage
142 Probability distribution storage
143 Model storage
150 Controller
151 Acquirer
152 Beam generator
153 Node joiner
154 Beam eliminator
155 Model generator
156 Outputter
200 Manufacturing device

1. A design device comprising:
an acquirer that acquires a design space in which a porous structure model is generated;
a beam generator that repeatedly generates a plurality of new beams branching from a node closer to a leading end of an existing beam while changing at least one of a beam length, a number of branches, or a rotation angle about an axis of the existing beam on a basis of a preset rule so that a plurality of beams included in a network structure of a porous structure are generated in the design space acquired by the acquirer;
a node joiner that selects a plurality of nodes that is not directly connected to each other through a beam, among the plurality of nodes generated by the beam generator, to join the selected nodes to generate a single node on a basis of a preset rule; and
a model generator that allows each beam included in a network structure obtained by the node joiner to be a volume to generate a porous structure model.

2. The design device according to claim 1, wherein
the beam generator generates a plurality of new beams so that each beam branching from an identical node three-dimensionally isotropically extends according to a number of branches, the number being set at each node of a beam.

3. The design device according to claim 1, wherein
in a case of generating a plurality of new beams branching from a node closer to a leading end of an existing beam, the beam generator may end generation of a beam at the node.

4. The design device according to claim 1, wherein in a case in which a node closer to a leading end of a new beam is present outside a design space, the beam generator ends generation of a beam at the node, and
the design device further includes a beam eliminator that eliminates a beam outside the design space, among beams that are generated by the beam generator and extend outside the design space.

5. The design device according to claim 1, wherein
in a case in which a node that satisfies a preset joinability condition is present within a search range from a node closer to a leading end of a new beam, the beam generator ends generation of a beam at the node closer to the leading end of the new beam, and
the node joiner joins a node at which generation of a beam is ended due to presence of a node that satisfies the joinability condition within the search range by the beam generator to a joinable node that is closest to the node.

6. The design device according to claim 5, wherein
the beam generator sets nodes at node numbers in order of generation of the nodes, and
the node joiner joins nodes at which generation of beams is ended due to presence of a node that satisfies a joinability condition within a search range in the beam generator to joinable nodes that are closest to the nodes in increasing order of the set node numbers.

7. The design device according to claim 1, wherein in a case in which a node density that is a number of nodes per unit volume within a search range set at a node closer to a leading end of a new beam is more than a node density threshold value, the beam generator ends generation of a beam at the node.

8. The design device according to claim 1, wherein the model generator transforms each beam into a beam-like member having an identical cross-sectional shape in at least an intermediate portion.

9. A design method that is executed by a design device, the design method comprising:
acquiring a design space in which a porous structure model is generated;
repeatedly generating a plurality of new beams branching from a node closer to a leading end of an existing beam while changing at least one of a beam length, a number of branches, or a rotation angle about an axis of the existing beam on a basis of a preset rule so that a plurality of beams included in a network structure of a porous structure are generated in the acquired design space;
selecting a plurality of nodes that is not directly connected to each other through a beam, among the generated plurality of nodes, to join the selected nodes to generate a single node on a basis of a preset rule; and
allowing each beam included in a network structure obtained to be a volume to generate a porous structure model.

10. A non-transitory computer-readable recording medium for recording a program that allows a computer to function as:
an acquiring means that acquires a design space in which a porous structure model is generated;
a beam generation means that repeatedly generates a plurality of new beams branching from a node closer to a leading end of an existing beam while changing at least one of a beam length, a number of branches, or a rotation angle about an axis of the existing beam on a basis of a preset rule so that a plurality of beams included in a network structure of a porous structure are generated in the design space acquired by the acquiring means;
a node joining means that selects a plurality of nodes that is not directly connected to each other through a beam, among the plurality of nodes generated by the beam generation means, to join the selected nodes to generate a single node on a basis of a preset rule; and
a model generation means that allows each beam included in a network structure obtained by the node joining means to be a volume to generate a porous structure model.

11. A porous structure comprising a plurality of beam-like members that is connected to each other through a node, wherein
each beam-like member is arranged so that an identical unit structure is prevented from being repeated in the porous structure, and
lengths of the beam-like members and a number of the beam-like members branching from the node are set to be distributed in ranges between upper and lower limits that are set, respectively.

12. The porous structure according to claim 11, wherein at least one of the plurality of nodes included in the porous structure are nodes that are arranged so that each beam-like member three-dimensionally isotropically extends from an identical node.

13. A method of manufacturing a porous structure, the method comprising manufacturing a porous structure on a basis of the porous structure model that is generated by the design device according to claim 1.
